(19) United States
(12) Patent Application Publication
(10) Pub. No.: US 2025/0292877 A1
(43) Pub. Date: Sep. 18, 2025

(54) SYSTEM AND METHOD FOR CONDUCTING A CLINICAL TRIAL

(71) Applicant: ACCUHEALTH TECHNOLOGIES INC., Atlanta, GA (US)

(72) Inventor: Elizabeth OFILI, Peachtree City, GA (US)

(21) Appl. No.: 19/059,642

(22) Filed: Feb. 21, 2025

Related U.S. Application Data
(60) Provisional application No. 63/556,576, filed on Feb. 22, 2024.

(51) Int. Cl. G16H 10/20; G16H 70/20

(52) U.S. Cl. CPC G16H 10/20; G16H 70/20

(57) ABSTRACT

A computer system programmed for providing access to at least one clinical investigation participant participating in at least one clinical investigation. In one embodiment, the system includes a clinical investigation provider module adapted for receiving, storing, and providing data in communication with at least one clinical investigation provider at the point of care, a clinical investigation participant module adapted for receiving, storing, and providing data in communication with the at least one clinical investigation participant, and, a clinical investigation data module adapted for receiving, storing, and providing data in communication with at least one clinical investigator. Each of the clinical investigation participant module, clinical investigation provider module, and clinical investigation data module is operatively associated with a corresponding one of the at least one clinical trial provider, at least one clinical investigation participant, and at least one clinical investigator through a network.

This application claims priority from U.S. Provisional Application No. 63/556,576, filed Feb. 22, 2024, which is incorporated herein by reference.

### FIELD

This application relates generally to the field of healthcare and conducting clinical trials.

### BACKGROUND

Decentralized Clinical Trials (DCT) are a rapidly growing market, bolstered by COVID-19 and the reimbursement of telemedicine and digital health technology (DHT). Participants prefer the convenience, and sponsors are getting on board. Some in the industry predict that this is the future of clinical trials and others like the FDA hope to expand access to under-represented populations through DCT and DHT trials. The major challenge is with the multiple data sources and challenges with data quality.

There are two major barriers relevant to this: 1) Lack of involvement of community physicians and their needs in the development and conduct of clinical trials. 2) Difficulty of recruiting and retaining an appropriate human subject population for specific clinical trials. These barriers disproportionately impact minorities and individuals with limited health literacy, in part due to a lack of cultural competence and language sensitivity among researchers. While the COVID-19 public health pandemic revealed the glaring health disparities affecting America's black and brown communities, with 2.4 to 7 times mortality rates compared to white Americans; the subsequent and pervasive misinformation/disinformation, exacerbated health disparities, and highlighted the urgent need for a resilient clinical trial enterprise, that include as investigators, frontline providers, who care for marginalized, rural and underserved populations.

Therefore, a need exists for the clinical trials enterprise to be sustainable; it follows that it must be worthwhile and practical for frontline healthcare providers who care for underrepresented populations, to participate as investigators.

### SUMMARY

One aspect of the present application relates to a computer system programmed for providing access to at least one clinical investigation participant participating in a clinical investigation. The computer system comprises a server comprising: (a) a clinical investigation provider module adapted for receiving, storing, and providing data in communication with at least one clinical investigation provider at assessment of clinical investigation eligibility, wherein the clinical investigation provider module comprises an algorithm for generating at least one clinical investigation eligibility decision for the at least one clinical trial participant at assessment of clinical investigation eligibility, wherein the algorithm is based at least in part on inclusion criteria and exclusion criteria for the clinical investigation; (b) a clinical investigation participant module adapted for receiving, storing, and providing data in communication with the at least one clinical investigation participant; and (c) a clinical investigation data module adapted for receiving, storing, and providing data in communication with at least one clinical investigator, wherein the clinical investigation data module is adapted for generating the inclusion criteria and exclusion criteria for the clinical investigation, wherein the server and each of the clinical investigation provider module, clinical investigation participant module, and clinical investigation data module are operatively associated with a corresponding one of the at least one clinical investigation provider, the at least one clinical investigation participant, and the at least one clinical investigator through a network computing system to perform for interacting with at least one user of the system programmed for providing access to at least one clinical investigation participant participating in the clinical investigation. In some embodiments, the clinical investigation is a clinical trial and the algorithm is further based on at least one of clinical trial participant-specific diagnostic guidelines, clinical trial participant-specific treatment guidelines, clinical trial participant-specific medication plans, and clinical trial participant-specific care pathways.

Another aspect of the present application relates to a method of using a computer system-programmed for providing access to at least one clinical trial participant participating in a clinical trial. The method comprises the steps of: (a) accessing the computer system through a network, the computer system comprising a server comprising: (i) a clinical trial provider module adapted for receiving, storing, and providing data in communication with at least one clinical trial provider at assessment of clinical trial eligibility, wherein the clinical trial provider module comprises an algorithm for generating at least one clinical trial eligibility decision for the at least one clinical trial participant at assessment of clinical trial eligibility, wherein the algorithm is based at least in part on inclusion criteria and exclusion criteria for the clinical trial; (ii) a clinical trial participant module adapted for receiving, storing, and providing data in communication with the at least one clinical trial participant; and (iii) a clinical trial data module adapted for receiving, storing, and providing data in communication with at least one clinical trial investigator, wherein the clinical trial data module is adapted for generating the inclusion criteria and exclusion criteria for the clinical trial, wherein the server and each of the clinical trial provider module, clinical trial participant module, and clinical trial data module are operatively associated with a corresponding one of the at least one clinical trial provider, the at least one clinical trial participant, and the at least one clinical trial investigator through a network computing system to perform for interacting with at least one user of the system programmed for providing access to at least one clinical trial participant participating in at least one clinical trial; (b) providing identification designating that the user is a clinical trial provider, clinical trial participant, or clinical trial investigator; and (c) performing at least one of the steps of: (i) providing data comprising at least one of information from electronic health records associated with the at least one clinical trial participant, information associated with at least one treatment compliance program for the at least one clinical trial, and information associated with the clinical target disease of the at least one clinical trial participant; (ii) receiving an alert when a proposed medication or proposed medication dosage conflicts with clinical trial participant-specific treatment guidelines, clinical trial participant-specific medication plans, and clinical trial participant-specific care pathways associated with the at least one clinical trial participant; (iii) receiving data for supporting clinical trial participant treatment compliance to clinical trial-specific treatment guidelines selected by at least one of the at least one clinical trial provider; (iv) providing data through the network from at least one of a health monitoring device, mobile electronic device, and user computer; (v) tracking use of clinical trial participant-specific forms by the at least one clinical trial participant; (vi) tracking communication between each of the at least one clinical trial participant and at least one of another clinical trial participant, the clinical trial provider; (vii) generating inclusion criteria and exclusion criteria associated with at least one clinical trial eligibility decision for a clinical trial selected by at least one of the at least one clinical trial provider, (viii) analyzing clinical trial participant-specific data; and (xi) receiving data associated with at least one clinical trial.

Another aspect of the present application relates to a method of using a computer system programmed for providing clinical to at least one clinical trial participant participating in a clinical trial. The method comprises the steps of: (a) accessing the computer system through a network; the computer system comprising a server comprising: (i) a clinical trial provider module adapted for receiving, storing, and providing data in communication with at least one clinical trial provider at assessment of clinical trial eligibility, wherein the clinical trial provider module comprises an algorithm for generating at least one clinical trial eligibility decision for the at least one clinical trial participant at assessment of clinical trial eligibility, wherein the algorithm is based at least in part on (1) inclusion criteria and exclusion criteria for the clinical trial; (ii) a clinical trial participant module adapted for receiving, storing, and providing data in communication with the at least one clinical trial participant; and (iii) a clinical trial data module adapted for receiving, storing, and providing data in communication with at least one clinical trial investigator, wherein the clinical trial data module is adapted for generating the inclusion criteria and exclusion criteria for the clinical trial, wherein the server and each of the clinical trial provider module, clinical trial participant module, and clinical trial data module are operatively associated with a corresponding one of the at least one clinical trial provider, the at least one clinical trial participant, and the at least one clinical trial investigator through a network computing system to perform for interacting with at least one user of the system programmed for providing access to at least one clinical trial participant participating in at least one clinical trial; (b) providing identification designating that the user is a clinical trial provider; and (c) performing at least one of the steps of: (i) providing data that comprises at least one of information from electronic health records associated with the at least one clinical trial participant, information associated with at least one treatment compliance program for the at least one clinical trial, and information associated with the clinical target disease of the at least one clinical trial participant; and (ii) receiving an alert when a proposed medication or proposed medication dosage conflicts with clinical trial participant-specific treatment guidelines, clinical trial participant-specific medication plans, and clinical trial participant-specific care pathways associated with the at least one clinical trial participant.

Another aspect of the present application relates to a software product that comprises a non-transitory computer-readable medium in which computer program instructions are stored. The instructions, when executed by a computing system, cause the computing system to perform the acts of: (a) receiving, storing, and providing data in communication with at least one clinical trial provider; (b) receiving, storing, and providing data in communication with at least one clinical trial participant participating in a clinical trial; (c) receiving, storing, and providing data in communication with at least one clinical trial investigator; (d) facilitating interactions between each of the at least one clinical trial provider, the at least one clinical trial participant, and the at least one clinical trial investigator; and (e) providing access to the at least one clinical trial participant, wherein the software comprises an algorithm for generating at least one clinical trial eligibility decision for the clinical trial for the at least one clinical trial participant at assessment of clinical trial eligibility and at least one social determinant of the clinical target disease of the at least one clinical trial participant, wherein the algorithm is based at least in part on (1) inclusion criteria and exclusion criteria for the clinical trial and (2) at least one of clinical trial participant-specific diagnostic guidelines, clinical trial participant-specific treatment guidelines, clinical trial participant-specific medication plans, and clinical trial participant-specific care pathways.

### BRIEF DESCRIPTION OF FIGURES

FIG. 1 shows an embodiment of the computer system of the present application. The computer system provides a platform for decentralized clinical investigations and clinical trials.

FIG. 2 shows an embodiment of the data network of the computer system present application.

FIG. 3 shows an embodiment of the information flow in the decentralized clinical trial platform herein.

FIG. 4 shows an embodiment of the quality control process in relation to the decentralized clinical trial platform herein.

FIG. 5 shows an embodiment of the security protection for the decentralized clinical trial platform herein.

FIG. 6 shows an embodiment of the information flow through the secured webportal for the decentralized clinical trial platform herein.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

Unless otherwise defined, all terms (including technical and scientific terms) used herein have the same meaning as commonly understood by one having ordinary skill in the art to which this application belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the application, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the application and the claims. In the following description, for purposes of explanation, numerous specific details are set forth in order to provide a thorough understanding of the present application. It will be evident, however, to one skilled in the art that the present application may be practiced without these specific details.

This application pertains to individuals in need of medical care who may present themselves to clinics and/or in doctor's offices with a set of symptoms to be included in a clinical trial, but would not typically be included in such trials because those individuals belong to marginalized populations. This application provides a method and system for including such individuals, who otherwise would not feature in clinical trials, within clinical trials, thereby ensuring that clinical trial results are more accurate and effective in assessing the impact of medical treatments.

### I. DEFINITIONS

As used herein, the term “clinical investigation” refers to a study in which one or more human subjects are prospectively investigated in a clinical setting. Clinical investigations include clinical trials in which one or more human subjects are assigned to one or more interventions (which may include placebo or other control) to evaluate the effects of those interventions on health-related biomedical or behavioral outcomes, and other clinical studies on human subjects. The term “clinical investigator” refers to a person who managers or monitors a clinical investigation, such as a clinical trial.

As used herein, the term “clinical investigation provider” or “clinical trial provider” refers to a healthcare provider that recommend or help a patient/individual to participate a clinical investigation or a clinical trial. A “clinical investigation provider” or “clinical trial provider” can be frontline healthcare providers, such as community physicians, who care for underrepresented populations in clinical investigations and clinical trials.

As used herein, the term “computer” refers to a machine, apparatus, or device that is capable of accepting and performing logic operations from software code. The term “application”, “software”, “software code” or “computer software” refers to any set of instructions operable to cause a computer to perform an operation. Software code may be operated on by a “rules engine” or processor. Thus, the methods and systems of the present application may be performed by a computer or computing device having a processor based on instructions received by computer applications and software.

The term “electronic device” as used herein is a type of computer comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, wearable computers such as Apple Watch, other smartwatches, Fitbit, other wearable fitness trackers, Google Glasses, and the like.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, laptops, tablet PCs including the iPad, cell phones including iOS phones made by Apple Inc., Android OS phones, Microsoft OS phones, Blackberry phones, Apple iPads, Anota digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e. a “wireless network”) which may include Wifi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VOIP) network. As used herein, the term “database” shall generally mean a digital collection of data or information. The present application uses novel methods and processes to store, link, and modify information such as digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e. information and data from a database may be recorded into a medium on a data store).

As used herein, the term “comorbidities” may refer to the simultaneous presence of two or more diseases or medical conditions in a clinical trial participant. In certain embodiments, co-morbidities may include one or more of, Active Chemotherapy, Advanced Directive Limiting Care, Alcoholism, Alzheimers Disease, Anemia, Angina Pectoris, Anticoagulant Therapy, Ascites within 30 Days, Asthma, Attention Deficit Disorder/Attention Deficit Hyperactivity Disorder, Bleeding Disorder, Cancer, Cardiopulmonary Resuscitation (CPR), Cerebrovascular Accident (CVA), Chronic Obstructive Pulmonary Disease (COPD), Chronic Renal Failure, Cirrhosis, Concurrent or Existence of Metastasis, Congestive Heart Failure, Congenital Anomalies, Coronary Artery Diseases, Coumadin Therapy, Current Smoker, Currently Receiving Chemotherapy for Cancer, Chronic Demyelinating Disease, Chronic Drug Abuse, Crohn's Disease, CVA/Hemiparesis (Stroke with Residual), Dementia, Diabetes Mellitus, Dialysis (Excludes Transplant Clinical trial participants), Disseminated Cancer, Documented History of Cirrhosis, Documented Prior History of Pulmonary Disease with Ongoing Active Treatment, Drug Use Disorder, DVT, Esophageal Varices, Functionally Dependent Health Status, Heart Disease, Hemophilia, History of Angina within Past 1 Month, History of Cardiac Surgery, History of Myocardial Infarction, History of Peripheral Vascular Disease (PVD), History of Psychiatric Disorders, HIV/AIDS, Hypertension, Insulin Dependent, Inflammatory Bowel Disease, Irritable Bowel Disease, Major Psychiatric Illness, Mental/Personality Disorder, Multiple Sclerosis, Myocardial Infarction (MI), Non-Insulin Dependent Diabetes Mellitus, Obesity, Pancreatitis, Parkinsons Disease, Peripheral Arterial Disease (PAD), Peripheral Vascular Disease (PVD), Pregnancy, Prehospital Cardiac Arrest with Resuscitative Efforts by Healthcare Provider, Prehospital CPR, Prematurity, Psychiatric Disorder, Pulmonary Embolus (PE), Renal Failure, Rheumatoid Arthritis, Routine Steroid Use, Seizures, Spinal Cord Injury, Steroid Use, Substance Abuse Disorder, Systemic Lupus Erythematous, Transplant, Ulcerative colitis, Undergoing Current Therapy, and Vascular Disease.

### II. A COMPUTER SYSTEM PROGRAMMED FOR PROVIDING ACCESS IN A CLINICAL INVESTIGATION

One aspect of the present application relates to a computer system programmed for providing access to a clinical investigation participant participating in a clinical investigation. The computer system comprises a server comprising: (a) a clinical investigation provider module adapted for receiving, storing, and providing data in communication with at least one clinical investigation provider at assessment of clinical investigation eligibility, wherein the clinical investigation provider module comprises an algorithm for generating at least one clinical investigation eligibility decision for the at least one clinical investigation participant at assessment of clinical investigation eligibility, wherein the algorithm is based at least in part on inclusion criteria and exclusion criteria for the clinical investigation; (b) a clinical investigation participant module adapted for receiving, storing, and providing data in communication with the at least one clinical investigation participant; and (c) a clinical investigation data module adapted for receiving, storing, and providing data in communication with at least one clinical investigator, wherein the clinical investigation data module is adapted for generating the inclusion criteria and exclusion criteria for the clinical trial, wherein the server and each of the clinical investigation provider module, clinical investigation participant module, and clinical investigation data module are operatively associated with a corresponding one of the at least one clinical investigation provider, the at least one clinical investigation participant, and the at least one clinical investigator through a network computing system to perform for interacting with at least one user of the system programmed for providing access to at least one clinical investigation participant participating in the clinical investigation. In some embodiments, the clinical investigation is a clinical trial.

In some embodiments, the present application relates to a computer system programmed for providing access to at least one clinical trial participant participating in a clinical trial, comprising a server comprising: (a) a clinical trial provider module adapted for receiving, storing, and providing data in communication with at least one clinical trial provider at assessment of clinical trial eligibility, wherein the clinical trial provider module comprises an algorithm for generating at least one clinical trial eligibility decision for the at least one clinical trial participant at assessment of clinical trial eligibility, wherein the algorithm is based at least in part on inclusion criteria and exclusion criteria for the clinical trial; (b) a clinical trial participant module adapted for receiving, storing, and providing data in communication with the at least one clinical trial participant; and (c) a clinical trial data module adapted for receiving, storing, and providing data in communication with at least one clinical trial investigator, wherein the clinical trial data module is adapted for generating the inclusion criteria and exclusion criteria for the clinical trial, wherein the server and each of the clinical trial provider module, clinical trial participant module, and clinical trial data module are operatively associated with a corresponding one of the at least one clinical trial provider, the at least one clinical trial participant, and the at least one clinical trial investigator through a network computing system to perform for interacting with at least one user of the system programmed for providing access to at least one clinical trial participant participating in the clinical trial.

In some embodiments, the algorithm is based at least in part on at least one of clinical trial participant-specific diagnostic guidelines, clinical trial participant-specific treatment guidelines, clinical trial participant-specific medication plans, and clinical trial participant-specific care pathways.

In some embodiments, the computer system includes a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

In some embodiments, the computer system includes a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

In some embodiments, the computer system includes special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

In some embodiments, the computer system includes a display controller coupled to the bus to control a display, such as a cathode ray tube (CRT), liquid crystal display (LCD) or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the application in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the application and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used.

In some embodiments, the computing system further includes clients and servers. A client and server are generally remote from each other and typically interact through a communication network or the cloud. The relationship of client and server arises by virtue of computer programs running on the respective computers and having a client server relationship to each other.

The different modules described herein in some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

The present application provides a HIPAA compliant clinical trial platform
application that incorporates several important features: First, the clinical trial platform application is a purpose built, co-design software as a service (SaaS), that will incorporate a Role-based access control (RBAC) single sign-on, site facing platform designed to streamline workflow and reduce site burden. Second, artificial intelligence (AI) machine learning (ML) and natural language processing (NLP) of unstructured data will automate technology enabled EHR recruitment and other study processes such as inclusion/exclusion criteria, and matching cases to controls. Third, the clinical trial platform application centers community partnered transparent data governance, with intelligent technologies for clinical investigation adaptive and “synthetic arms” study design. Fourth, the clinical trial platform application adopts a quality by design risk-based monitoring to improve trial efficiency, real time data quality control, and workflow integration, for seamless study management, and a transparent e-Consent process. Fifth, the clinical trial platform application protocol based training, and virtual collaboratory for continuous learning, provides ongoing mentorship and peer to peer support to site clinical investigation coordinators. Sixth, the conceptual model integrates workflows for a care-to-research continuum, designed to improve clinical trial access for small to medium practices and the diverse patient populations they serve.

### Clinical Investigation Provider Module

The clinical investigation or clinical trial provider module comprises clinical information that can be accessed in a manner reflecting the state of record keeping in each individual entity (e.g., clinical office). In clinical offices having relatively modern electronic health record systems, the desired clinical information can be collected from the electronic health record (“EHR”) of each patient for each encounter. In clinical offices without comprehensive EHR systems, or in those using different data formats, individually crafted interfaces or other means specific to the particular entities or data types can be used to collect information. For example, lab, pathology reports and imaging tests results can be accessed by interfaces to the clinical trial provider module used to maintain this data via a secure webportal. Internet connections or mobile network access will enable the secure webportal to be used across a number of clinical trial providers. The clinical information may also include payor claims data, questionnaire data and clinical data. Relevant information from the clinical investigation/clinical trial provider module can be accessed through pharmacy systems, such as those maintained by hospitals or third parties. Admission history and physical examination and discharge summaries are also retained in the clinical trial provider module. In some embodiments, the clinical trial provider module is connected to labs, imaging centers, outpatient surgery centers, and/or other entities.

The clinical trial data stored in the clinical trial provider module includes non-electronic information, such as non-electronic records gathered from physician's notes and/or other written records from a physician's office. The non-electronic records may include information that is not available from the EHR, payor claims data, questionnaire data and clinical data, but which is recorded in notebooks, on charts, etc. Such non-electronic records may be scanned and read by artificial intelligence/machine learning software that can then store the note as information in the clinical trial provider module. Such information may be relatively limited and, for example, consists of results of certain tests done in the physician's office, e.g., EKG's, cardiac stress tests, echocardiogram tests, EEG's, EMG's, nerve conduction studies, and ultrasound tests. To facilitate the clinical trial conduct, the clinical trial provider module systematizes and standardizes the information required for the clinical trial and makes it accessible through the secure webportal.

Clinical investigation/trial eligibility is determined by inclusion criteria and exclusion criteria. These define the patient population under investigation. These criteria are often tailored to allow assessments of the effectiveness of a treatment in a well-defined population. Inclusion criteria specify the characteristics required for study entry, such as stage of disease or specific pathophysiological characteristics. They typically identify a population in which it is expected that the effect of the drug can be shown. A control group is the group of participants that receives standard of care treatment. The control group may also be comprised of healthy volunteers. An experimental group is the group of participants in a study that receive the experimental treatment or study intervention. A treatment arm is a group or subgroup of participants in a clinical trial. Each group receives a specific intervention, study drug dose, or sometimes no intervention, according to the study protocol.

An example is identifying patients with a specific mutation that is targeted by the treatment, where the drug is likely to be effective only in those patients with the disease who have the mutation. More broadly, it is usual to not only include patients who have the disease to be treated, but those who also have a threshold severity of disease and do not have certain other conditions, or who are not using medications that could mask the effect. Exclusion criteria specify characteristics that disqualify patients from participation and often include factors such as comorbidities or concomitant treatment or factors that could mask the effect of the intervention. However, if these criteria exclude a subgroup that will eventually receive the drug once approved (e.g., people with comorbidities), relevant effects of the drug on that subgroup will not be detected. Broader inclusion criteria and less-restrictive exclusion criteria will lead to a study that provides more information about the product's effects in the population most likely to use the product if it is approved. This also includes record of informed consent, which requires that a clinical investigation/trial participant has learned the key facts about a clinical study, including the possible risks and benefits, and agrees to take part in it.

Clinical investigation/trial participant-specific diagnostic guidelines are diagnostic criteria. Diagnostic criteria are a set of signs, symptoms, and tests used to determine the diagnosis of a person. Diagnostic criteria may be broad to accurately identify as many people with the condition as possible if the signs and symptoms of a disease, or symptom or progression severity, varies from person to person. Alternatively, there may be a single diagnostic test, such as a genetic test, that can confirm or establish a diagnosis. Ideally diagnostic criteria are validated through research studies.

Clinical investigation/trial participant-specific treatment guidelines refer to the parameters for the group of patients who are intended to participate in the clinical investigation/trial. Each group or subgroup of participants in a clinical investigation/trial receives a specific intervention, study drug dose, or sometimes no intervention, according to the study protocol. Outcome measures that are a planned measurement described in the protocol that is used to determine the effect of interventions on participants in a clinical investigation/trial. For observational studies, a measurement or observation is used to describe patterns of diseases or traits, or associations with exposures, risk factors, or treatment.

Clinical investigation/trial participant-specific medication plans encompass the particular medical intervention being studied in the clinical investigation/trial. Typically, there is a treatment, drug or procedure that is being studied in the clinical investigation/trial. This term is typically used when compared to a control or standard of care treatment arm. An “Interventional” trial is a term used to describe clinical investigation/trials studying a treatment, drug or procedure. Alternatively, in an observational study, investigators assess health outcomes in groups of participants according to an investigation plan or protocol. Participants may receive diagnostic or other types of interventions as part of their routine medical care, but the investigator does not assign participants to specific interventions or treatments.

Clinical investigation/trial participant-specific care pathways refer to those parameters that are being impacted in clinical investigation/trial participants or are of interest to the clinical investigators. Such information may include baseline characteristics such as data collected at the beginning of a clinical study for all participants and for each arm or comparison group. These data may include demographics, such as age, race, and gender, and any study-specific measures (e.g. systolic blood pressure, prior antidepressant treatment). Adverse events are also recorded; such as any undesirable experience associated with a drug or procedure, like a side effect or negative reaction. Adverse events can range from mild to severe. Serious adverse events are those that can cause temporary or permanent disability and may result in hospitalization or death.

A clinical investigation/trial participant module comprises information that is either input or collected directly from the clinical investigation/trial participant. This may include information provided by health monitoring devices worn by the patient that are connected to the clinical trial participant module via, for example, a mobile wireless network or the internet. In some embodiments, the clinical investigation/trial participant module contains questionnaires and surveys for clinical investigation/trial participants.

A clinical investigation/trial data module comprises a repository of all data collected during the course of the clinical investigation/trial from all sources, including both clinical investigation/trial providers and clinical investigation/trial participant. Data from questionnaires and surveys from the clinical investigation/trial participants may also be stored in the clinical investigation/trial participant module.

A clinical trial investigator is an individual or entity (e.g., medical agency) that is receiving and analyzing the results of the clinical trial. The clinical trial investigator may be the clinical trial sponsor (e.g., a pharmaceutical company) or an individual working for the clinical trial sponsor, or a third party reviewer (such as an FDA reviewer assessing the results of a clinical trial).

The present application, in one aspect, relates to a computer system programmed for providing clinical investigation access to at least one clinical trial having at least one clinical target disease treatment for study; in certain embodiments, comorbidities may also be included in study of the clinical trial. In one embodiment, the system includes a clinical trial provider module adapted for receiving, storing, and providing data in communication with at least one clinical trial provider at the point of care, a clinical trial participant module adapted for receiving, storing, and providing data in communication with the at least one clinical trial participant, and a clinical trial data module adapted for receiving, storing, and providing data in communication with at least one clinical trial investigator. Each of the clinical trial provider module, clinical trial participant module, and clinical trial data module is operatively associated with a corresponding one of the at least one clinical trial provider, at least one clinical trial participant, and at least one clinical trial investigator through a data network.

In one embodiment, the system is further programmed for performing at least one of the functions of providing data including at least one of information from electronic health records associated with the at least one clinical trial participant, information associated with at least one treatment compliance program for treating the at least one clinical target disease, and information associated with the clinical target disease of the at least one clinical trial participant, and receiving data including a decision support algorithm for generating at least one clinical trial eligibility decision for the at least one clinical trial participant. The decision support algorithm includes the inclusion criteria and exclusion criteria for the clinical trial. In some embodiments, the decision support algorithm further includes at least a part of the clinical trial participant-specific national diagnostic guidelines, the clinical trial participant-specific national treatment guidelines, the clinical trial participant-specific medication plans, and/or the clinical trial participant-specific care pathways for the clinical trial. The possible functions also include receiving an alert when a proposed medication or proposed medication dosage conflicts with a current clinical trial medication or current clinical trial medication dosage associated with the at least one clinical trial participant, receiving data for supporting clinical trial participant treatment compliance to clinical target clinical trial participant-specific treatment guidelines selected by at least one of the at least one clinical trial provider, providing data through the network from at least one of a medical monitoring device, mobile electronic device, and user computer, tracking use of clinical trial participant-specific curriculum by the at least one clinical trial participant, tracking communication between each of the at least one clinical trial participant and at least one of another clinical trial participant, the clinical trial provider, generating cost-benefit information associated with at least one treatment decision selected by at least one of the at least one clinical trial provider, analyzing clinical trial participant-specific data, and receiving data associated with at least one clinical health trial. Each of the clinical trial provider module, clinical trial participant module, and clinical trial data module is further operatively associated with at least one data storage means through the network, where the at least one data storage means is adapted for storing data saved by the at least one corresponding clinical trial provider, at least one clinical trial participant, and at least one clinical trial investigator.

In one embodiment, the at least one clinical target disease is heart disease, diabetes, obesity, or asthma, and the network is at least one of the internet and a mobile electronic device network. The at least one clinical trial provider is a physician, physician assistant, nurse, or nurse practitioner, or a clinical research coordinator. Clinical trial provider as understood herein encompasses any individual who fulfils a role in conducting clinical trials. In some embodiments, the computer system of the present application provides needs-based research coordinator training and/or workshop. The data received by at least one of the clinical trial provider module 126 and includes at least one of information from electronic health records associated with the at least one clinical trial participant, information associated with treatment compliance programs for treating the at least one clinical target disease, and information associated with the clinical target disease of the at least one clinical trial participant. The data provided by the clinical trial provider module includes at least one decision support algorithm for generating at least one clinical trial eligibility decision for the at least one clinical trial participant at the point of care. The at least one algorithm is based at least in part on at least one of clinical trial participant-specific national diagnostic guidelines, clinical trial participant-specific national treatment guidelines, clinical trial participant-specific medication plans, and clinical care pathways. The clinical trial provider module is further adapted for providing an alert to the at least one clinical trial provider when a proposed medication or proposed medication dosage conflicts with a current clinical trial medication or current clinical trial medication dosage associated with the at least one clinical trial participant. The data provided includes information for supporting clinical trial participant treatment compliance to clinical target clinical trial participant-specific treatment guidelines selected by at least one of the at least one clinical trial provider. The data received by the clinical trial participant module includes information provided by the at least one clinical trial participant through the network from at least one of a health monitoring device, mobile electronic device, and user computer. The data provided by the clinical trial participant module includes at least one representation of the clinical target disease of the at least one clinical trial participant and the at least one representation includes at least one of graphs and charts associated with the clinical target disease of the at least one clinical trial participant.

In one embodiment, the clinical trial participant module is further adapted for tracking use of clinical trial participant-specific curriculum by the at least one clinical trial participant and is further adapted for tracking communication between the at least one clinical trial participant and at least one of another clinical trial participant, and the at least one clinical trial provider. The clinical trial data module is further adapted for generating cost-benefit information associated with at least one clinical trial eligibility decision selected by at least one of the at least one clinical trial provider, analyzing clinical trial participant-specific data, and receiving data associated with at least one clinical health trial. Each of the clinical trial provider module, clinical trial participant module, and clinical trial data module is operatively associated with a graphical user interface including controls for selectively receiving, storing, and providing data in the system in response to an action by at least one of the clinical trial provider, the at least clinical trial participant, and the at least one clinical trial investigator. In one embodiment, the at least one health monitoring device is a heart rate monitor, blood pressure meter, peak flow meter, pulse oximeter, pedometer, weighing scale, or glucometer.

In another aspect, the present application relates to a computer system programmed for providing clinical trial supervision to at least one clinical trial participant having at least one clinical target disease. In one embodiment, the system includes an application programming interface (API), for generating at least one representation of data to at least one user, where the at least one user is a clinical trial provider, clinical trial participant, or clinical trial investigator, wherein the interface having interactive user controls for selectively receiving, storing, and providing data in response to actions by the at least one user, an analysis capability for analyzing data, a communication capability for sending and receiving data to and from the at least one user and programmed for controlling the functions performed by each of the API, graphical user interface, analysis tools, communication tools, and a storage capability for storing data saved by the at least one user. The data includes information associated with at least one of the at least one clinical trial participant and at least one clinical target disease, and at least one of the API, graphical user interface, analysis tools, communication tools, and is operatively associated with the at least one user through a network. The data includes at least one decision support algorithm for generating at least one clinical trial eligibility decision for the at least one clinical trial participant at the point of care. The interactive user controls are graphical user interface controls displayed on at least one of a user computer or a mobile electronic device. At least one representation is generated by the representation means and includes at least one of graphs and charts associated with the clinical target disease of the at least one clinical trial participant, and is displayed on at least one of a user computer or a mobile electronic device.

In one embodiment, the system further includes an access control means for controlling access to at least one of the representation means, interface means, analysis means, communication means, and storage means, based upon the identity of the at least one user as a clinical trial provider, clinical trial participant, or clinical trial investigator. The network is the internet or a mobile electronic device network and at least one of the representation means, interface means, analysis means, and communication means is operatively associated with at least one of a user computer or user mobile electronic device through the network, where at least one of the representation means, interface means, analysis means, and communication means includes a web-based application.

### III. A METHOD OF USING A COMPUTER SYSTEM-PROGRAMMED FOR PROVIDING ACCESS IN A CLINICAL INVESTIGATION

Another aspect of the application relates to method of using a computer system-programmed for providing access to at least one clinical investigation/clinical trial participant participating in a clinical investigation or clinical trial, comprising the steps of: (a) accessing the computer system through a network, the computer system comprising a server comprising: (i) a clinical investigation/trial provider module adapted for receiving, storing, and providing data in communication with at least one clinical investigation/trial provider at assessment of clinical investigation/trial eligibility, wherein the clinical investigation/trial provider module comprises an algorithm for generating at least one clinical investigation/trial eligibility decision for the at least one clinical investigation/trial participant at assessment of clinical investigation/trial eligibility, wherein the algorithm is based at least in part on inclusion criteria and exclusion criteria for the clinical investigation/trial; (iii) a clinical investigation/trial participant module adapted for receiving, storing, and providing data in communication with the at least one clinical investigation/trial participant; and (iv) a clinical investigation/trial data module adapted for receiving, storing, and providing data in communication with at least one clinical investigator/clinical trial investigator, wherein the clinical investigation/trial data module is adapted for generating inclusion criteria and exclusion criteria for the clinical investigation/trial, wherein the server and each of the clinical investigation/trial provider module, clinical investigation/trial participant module, and clinical investigation/trial data module are operatively associated with a corresponding one of the at least one clinical investigation/trial provider, the at least one clinical investigation/trial participant, and the at least one clinical investigator or clinical trial investigator through a network computing system to perform for interacting with at least one user of the system programmed for providing access to at least one clinical investigation/trial participant participating in at least one clinical investigation/trial; (b) providing identification designating that the user is a clinical investigation/trial provider, clinical investigation/trial participant, or clinal investigator/clinical trial investigator; and (c) performing at least one of the steps of: (i) providing data comprising at least one of information from electronic health records associated with the at least one clinical investigation/trial participant, information associated with at least one treatment compliance program for the at least one clinical investigation/trial, and information associated with the clinical target disease of the at least one clinical investigation/trial participant; (ii) receiving an alert when a proposed medication or proposed medication dosage conflicts with clinical investigation/trial participant-specific treatment guidelines, clinical investigation/trial participant-specific medication plans, and clinical investigation/trial participant-specific care pathways associated with the at least one clinical investigation/trial participant; (iii) receiving data for supporting clinical investigation/trial participant treatment compliance to clinical investigation/trial-specific treatment guidelines selected by at least one of the at least one clinical investigation/trial provider; (iv) providing data through the network from at least one of a health monitoring device, mobile electronic device, and user computer; (v) tracking use of clinical investigation/trial participant-specific forms by the at least one clinical investigation/trial participant; (vi) tracking communication between each of the at least one clinical investigation/trial participant and at least one of another clinical investigation/trial participant, the clinical investigation/trial provider; (vii) generating inclusion criteria and exclusion criteria associated with at least one clinical investigation/trial eligibility decision for a clinical investigation/trial selected by at least one of the at least one clinical investigation/trial provider; (viii) analyzing clinical investigation/trial participant-specific data; and (xi) receiving data associated with at least one clinical investigation/trial. In some embodiments, the algorithm is further based on at least part of a clinical investigation/trial participant-specific diagnostic guidelines, clinical investigation/trial participant-specific treatment guidelines, clinical investigation/trial participant-specific medication plans, and/or clinical investigation/trial participant-specific care pathways for the clinical investigation/trial.

Another aspect of the application is a method of using a computer system programmed for providing access to at least one clinical investigation/trial participant participating in a clinical investigation/trial, comprising the steps of: (a) accessing the computer system through a network; the computer system comprising a server comprising: (i) a clinical investigation/trial provider module adapted for receiving, storing, and providing data in communication with at least one clinical investigation/trial provider at assessment of clinical investigation/trial eligibility, wherein the clinical investigation/trial provider module comprises an algorithm for generating at least one clinical investigation/trial eligibility decision for the at least one clinical trial participant at assessment of clinical investigation/trial eligibility, wherein the algorithm is based on inclusion criteria and exclusion criteria for the clinical investigation/trial; (iii) a clinical trial participant module adapted for receiving, storing, and providing data in communication with the at least one clinical investigation/trial participant; and (iv) a clinical investigation/trial data module adapted for receiving, storing, and providing data in communication with at least one clinical investigator/clinical trial investigator, wherein the clinical investigation/trial data module is adapted for generating inclusion criteria and exclusion criteria for the clinical investigation/trial, wherein the server and each of the clinical investigation/trial provider module, clinical investigation/trial participant module, and clinical investigation/trial data module are operatively associated with a corresponding one of the at least one clinical investigation/trial provider, the at least one clinical trial participant, and the at least one clinical investigator/clinical trial investigator through a network computing system to perform for interacting with at least one user of the system programmed for providing access to at least one clinical investigation/trial participant participating in at least one clinical investigation/trial; (b) providing identification designating that the user is a clinical investigation/trial provider; and (c) performing at least one of the steps of: (i) providing data that comprises at least one of information from electronic health records associated with the at least one clinical investigation/trial participant, information associated with at least one treatment compliance program for the at least one clinical investigation/trial, and information associated with the clinical target disease of the at least one clinical investigation/trial participant; (ii) receiving an alert when a proposed medication or proposed medication dosage conflicts with clinical investigation/trial participant-specific treatment guidelines, clinical investigation/trial participant-specific medication plans, and clinical investigation/trial participant-specific care pathways associated with the at least one clinical investigation/trial participant. In some embodiments, the algorithm is further based on at least part of a clinical investigation/trial participant-specific diagnostic guidelines, clinical investigation/trial participant-specific treatment guidelines, clinical investigation/trial participant-specific medication plans, and/or clinical investigation/trial participant-specific care pathways for the clinical investigation/trial.

In yet another aspect, the present application relates to a method of using a computer system programmed for providing care support to at least one clinical investigation/trial participant having at least one clinical target disease. In one embodiment, the method includes the steps of accessing the computer system through a network and providing identification designating that the user is a clinical investigation/trial provider, clinical investigation/trial participant, or clinical investigator or clinical trial investigation/. The method also includes performing at least one of the steps of providing data including at least one of information from electronic health records associated with the at least one clinical investigation/trial participant, information associated with at least one treatment compliance program for treating the at least one clinical target disease, and information associated with the clinical target disease of the at least one clinical investigation/trial participant, receiving data including at least one decision support algorithm for generating at least one clinical investigation/trial eligibility decision for the at least one clinical investigation/trial participant at the point of care, where the at least one decision support algorithm is based on (1) inclusion criteria and exclusion criteria for a clinical investigation/trial and (2) at least one of clinical investigation/trial participant-specific national diagnostic guidelines, clinical investigation/trial participant-specific national treatment guidelines, clinical investigation/trial participant-specific medication plans, and clinical care pathways for the clinical investigation/trial, receiving an alert when a proposed clinical trial medication or proposed clinical investigation/trial medication dosage conflicts with a current medication or current medication dosage associated with the at least one clinical investigation/trial participant, receiving data for supporting clinical investigation/trial participant treatment compliance to clinical target clinical investigation/trial participant-specific treatment guidelines selected by at least one of the at least one clinical trial provider, providing data through the network from at least one of a health monitoring device, mobile electronic device, and user computer, tracking use of clinical investigation/trial participant-specific curriculum by the at least one clinical investigation/trial participant, tracking communication between each of the at least one clinical investigation/trial participant and at least one of another clinical investigation/trial participant, the clinical investigation/trial provider, generating cost-benefit information associated with at least one treatment decision selected by at least one of the at least one clinical investigation/trial provider, analyzing clinical investigation/trial participant-specific data, and, receiving data associated with the clinical investigation/trial.

In yet another aspect, the present application relates to a method of using a computer system programmed for providing care support to at least one clinical investigation/trial participant having at least one clinical target disease. In one embodiment, the method includes the steps of accessing the computer system through a network and providing identification designating that the user is a clinical investigation/trial provider. The method also includes performing at least one of the steps of providing data that includes at least one of information from electronic health records associated with the at least one clinical investigation/trial participant, information associated with at least one treatment compliance program for treating the at least one clinical target disease, and information associated with the clinical target disease of the at least one clinical investigation/trial participant, receiving data that includes a decision support algorithm adapted for generating at least one clinical investigation/trial eligibility decision for the at least one clinical investigation/trial participant at the point of care, where the at least one decision support algorithm includes inclusion criteria and exclusion criteria for a clinical investigation/trial and at least one of clinical investigation/trial participant-specific national diagnostic guidelines, clinical investigation/trial participant-specific national treatment guidelines, clinical investigation/trial participant-specific medication plans, and clinical care pathways for the clinical investigation/trial and, receiving an alert when a proposed medication or proposed medication dosage conflicts with a current medication or current medication dosage associated with the at least one clinical investigation/trial participant. In one embodiment, the method further includes the step of generating at least one clinical investigation/trial eligibility decision for the at least one clinical investigation/trial participant at the point of care.

In yet another aspect, the present application relates to a method of using a computer system programmed for providing care support to at least one clinical investigation/trial participant having at least one clinical target disease. In one embodiment, the method includes the steps of accessing the computer system through a network connection. The method further includes performing at least one of the steps of receiving data for supporting clinical investigation/trial participant treatment compliance to clinical target clinical investigation/trial participant-specific treatment guidelines selected by at least one of the clinical trial provider, tracking use of clinical investigation/trial participant-specific curriculum by the at least one clinical investigation/trial participant, and, tracking communication between each of the at least one clinical investigation/trial participant and at least one of another clinical investigation/trial participant, the at least one clinical investigation/trial provider. In one embodiment, the method further includes the step of generating at least one decision associated with clinical trial participant treatment compliance for the at least one clinical investigation/trial participant.

In yet another aspect, the present application relates to a method for using a computer system programmed for providing care support for at least one clinical investigation/trial participant having at least one clinical target disease. In one embodiment, the method includes the steps of accessing the computer system through a network and providing identification designating that the user is the at least one clinical investigation/trial participant. The method also includes performing at least one of the steps of providing data associated with the clinical target disease of the at least one clinical investigation/trial participant through the network from at least one of a health monitoring device, mobile electronic device, and user computer, viewing at least one representation of the clinical target disease of the at least one clinical investigation/trial participant, viewing clinical investigation/trial participant-specific curriculum, and, communicating with at least one of another clinical investigation/trial participant, the at least one clinical investigation/trial provider, through the network. In one embodiment, the method further includes at least one of the steps of receiving, generating, and sending at least one of an SMS text message, electronic mail message, and social networking message, where the step of communicating with at least one of another clinical investigation/trial participant, the at least one clinical investigation/trial provider, and utilizes the network connection.

In yet another aspect, the present application relates to a method for using a computer system programmed for providing care support to at least one clinical investigation/trial participant having at least one clinical target disease, where the system includes a clinical investigation/trial provider module adapted for receiving, storing, and providing data in communication with at least one clinical investigation/trial provider, a clinical investigation/trial participant module adapted for receiving, storing, and providing data in communication with the at least one clinical investigation/trial participant, and a clinical investigation/trial data module adapted for receiving, storing, and providing data in communication with at least one clinical investigator/clinical trial investigator, and where each of the clinical trial investigation/provider module, clinical investigation/trial participant module, and clinical investigation/trial data module is operatively associated with the corresponding at least one clinical investigation/trial provider, at least one clinical investigation/trial participant and at least one clinical investigator/clinical trial investigator through a network connection. In one embodiment, the method includes at least one of the steps of causing the clinical investigation/trial provider module to perform at least one of the functions of receiving data, storing data, and providing data, causing the clinical investigation/trial participant module to perform at least one of receiving data, storing data, and providing data, and, causing the clinical investigation/trial data module to perform at least one of receiving data, storing data, and providing data, where the data includes at least one decision support algorithm for generating at least one clinical investigation/trial eligibility decision for the at least one clinical investigation/trial participant at the point of care and at least one visual representation of the clinical target disease of the at least one clinical investigation/trial participant.

In yet another aspect, the present application relates to a method for using a computer system that is programmed for providing care support to at least one clinical investigation/trial participant having at least one clinical target disease, where the system includes a representation means for generating at least one representation of data to at least one user, and where the at least one user is a clinical investigation/trial provider, clinical investigation/trial participant, or clinical investigator/clinical trial investigator. The computer system also includes an interface means having interactive user controls for manipulating data in response to an action by the at least one user, an analysis means for analyzing data, a communication means for sending and receiving data to and from the at least one user, a storage means for storing data saved by the at least one user, and a network server in communication with and programmed for controlling the functions performed by each of the representation means, interface means, analysis means, communication means, and storage means. At least one of the representation means, interface means, analysis means, and communication means is operatively associated with the at least one user through a network. In one embodiment, the method includes at least one of the steps of causing the representation means to generate at least one visual representation of data, causing the interactive user controls of the interface means to selectively receive, store, and provide data, causing the analysis means to analyze data, causing the communication means to send and receive data, and, causing the network server to communicate with at least one of the representation means, interface means, analysis means, communication means, and storage means, and to control each function of the representation means, interface means, analysis means, communication means, and storage means. The data includes information associated with at least one of the at least one clinical trial participant and at least one clinical target disease.

### IV. A SOFTWARE PRODUCT COMPRISING A NON-TRANSITORY COMPUTER-READABLE MEDIUM IN WHICH COMPUTER PROGRAM INSTRUCTIONS ARE STORED

Another aspect of the application relates to a software product comprising a non-transitory computer-readable medium in which computer program instructions are stored, said instructions, when executed by a computing system, cause the computing system to perform the acts of: (a) receiving, storing, and providing data in communication with at least one clinical investigation/trial provider; (c) receiving, storing, and providing data in communication with at least one clinical investigation/trial participant participating in a clinical investigation/trial; (d) receiving, storing, and providing data in communication with at least one clinical investigator/clinical trial investigator; (e) facilitating interactions between each of the at least one clinical investigation/trial provider, the at least one clinical investigation/trial participant, and the at least one clinical investigator/clinical trial investigator; and (f) providing clinical to the at least one clinical investigation/trial participant, wherein the software comprises an algorithm for generating at least one clinical investigation/trial eligibility decision for the at least one clinical investigation/trial participant at assessment of clinical investigation/trial eligibility and at least one social determinant of the clinical target disease of the at least one clinical investigation/trial participant, wherein the algorithm is based on inclusion criteria and exclusion criteria for the clinical trial. In some embodiments, the algorithm is further based on at least in part on at least one of clinical investigation/trial participant-specific diagnostic guidelines, clinical investigation/trial participant-specific treatment guidelines, clinical investigation/trial participant-specific medication plans, and clinical investigation/trial participant-specific care pathways for the clinical trial.

In yet another aspect, the present invention relates to software stored on a computer-readable medium for causing a computing system to perform functions for interacting with at least one user of a system, where the system is programmed for providing care support to at least one clinical investigation/trial participant having at least one clinical target disease. In one embodiment, the functions include receiving, storing, and providing data in communication with at least one clinical investigation/trial provider, receiving, storing, and providing data in communication with at least one clinical trial participant, and, receiving, storing, and providing data in communication with at least one clinical investigator/clinical trial investigator. The data includes a decision support algorithm for generating at least one care decision for the at least one clinical investigation/trial participant at the point of care and at least one visual representation of the clinical target disease of the at least one clinical investigation/trial participant.

In yet another aspect, the present invention relates to software stored on a computer-readable medium for causing a computing system to perform functions for interacting with at least one user of a system programmed for providing care support to at least one clinical investigation/trial participant having at least one clinical target disease. In one embodiment, the functions include generating at least one visual representation of data to the at least one user, selectively receiving, storing, and providing data in response to an action by the at least one user, analyzing data, sending data, and receiving data to and from the at least one user, and, storing data saved by the at least one user. The data includes information associated with at least one of the at least one clinical trial participant and at least one clinical target disease, and the communication occurs through a network.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the application may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

In some embodiments, the tangible non-transitory computer readable storage medium described herein, further comprises instructions that, when executed by a computer processor, cause the processor to: (i) enable one or more computing devices in data communication with each other, each device having one or more computer processors, a data communication connection, and one or more tangible non-transitory computer-readable media accessible by the one or more computer processors, (ii) store a personal information database; and (iii) inputting into a machine learning module, wherein the personal information database and the machine learning module are each stored in the one or more tangible non-transitory computer-readable media.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the particular methods and/or corresponding acts in support of steps and corresponding functions described herein, may also be utilized to implement corresponding software structures and algorithms, and equivalents thereof. The processes described in this specification can be performed by one or more programmable processors (computing device processors) executing one or more computer applications or programs to perform functions by operating on input data and generating output.

Stored on any one or on a combination of computer readable media, the present application includes software for controlling the computer system, for driving a device or devices for implementing the application, and for enabling the computer system to interact with a human user. Such software may include, but is not limited to, device drivers, operating systems, development tools, and applications software. Such computer readable media further includes the computer program product of the present application for performing all or a portion (if processing is distributed) of the processing performed in implementing the application.

The computer code or software code of the present application may be any interpretable or executable code mechanism, including but not limited to scripts, interpretable programs, dynamic link libraries (DLLs), Java classes, and complete executable programs. Moreover, parts of the processing of the present application may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present application remotely into a dynamic memory and send the instructions over the air (e.g., through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device or client device such as a personal digital assistant (PDA), laptop computer, tablet computer, smartphone, or cellular telephone. The LAN communications network and the other communications networks such as cellular wireless and wifi networks may use electrical, electromagnetic or optical signals that carry digital data streams. The processor system can transmit notifications and receive data, including program code, through the network(s), the network link and the communication interface.

The present application is further illustrated by the following examples that should not be construed as limiting. The contents of all references, patents, and published patent applications cited throughout this application, as well as the Figures and Tables, are incorporated herein by reference.

### EXAMPLES

### Example 1

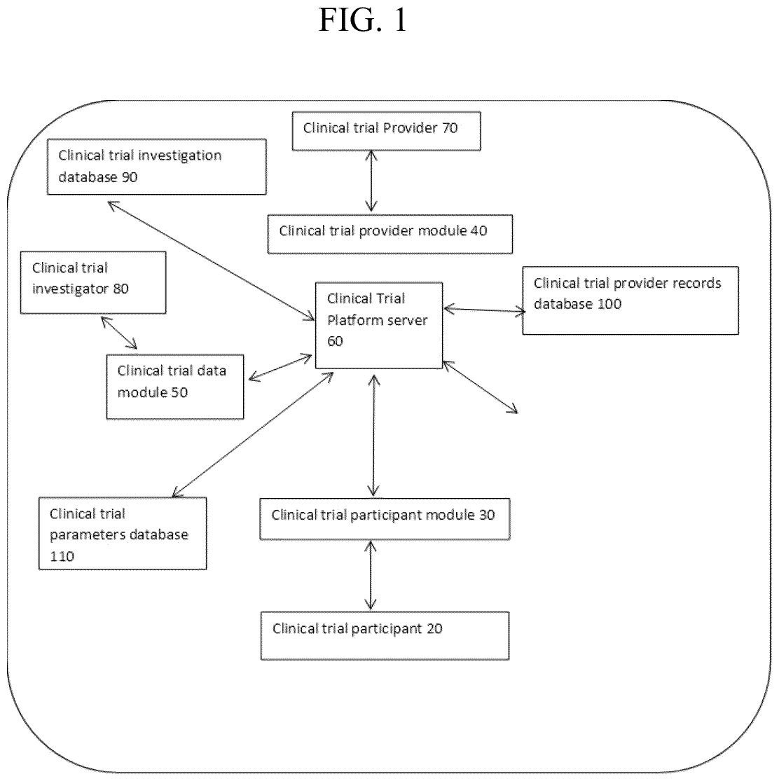

FIG. 1 shows schematically a computer system 10 programmed for providing care support to at least one clinical trial participant 20 having at least one clinical target disease. In operation, the system 10 enables close monitoring of the health of a clinical trial participant and access is through multiple devices, such as a computer, cell phone, or health monitoring device. Easy communication is also allowed between clinical trial participants and physicians, and early detection potential is provided for detecting deteriorating conditions, by physician intervention. Treatment compliance and adherence is promoted, and dynamically linked clinical trial-specific health guidelines are also provided to the clinical trial participant.

The system 10 can be a web-based application such as a publicly available or proprietary web page, and includes a clinical trial participant module 30, clinical trial provider module 40, and clinical trial data module 50, which here is to a clinical trial platform server 60 that provides communication for a data network. The users shown are a clinical trial participant 20, clinical trial provider 70, and a clinical trial investigator 80. The clinical trial participant 20 is a clinical trial participant having at least one clinical target disease, such as diabetes, heart disease, or asthma. The clinical trial provider 70 is a physician, physician assistant, nurse practitioner, or nurse, for example, who works with the clinical trial participant 20 to study the clinical target disease directly at the point of care during the clinical trial. The clinical trial investigator 80 can be a clinical trial data investigator that works to collect and analyze data from clinical trial participants in clinical trials.

The clinical trial participant module 30 is operatively associated with a clinical trial participant 20, the clinical trial data module 50 is operatively associated with a clinical trial investigator 80, and the clinical trial provider module 40 is operatively associated with a clinical trial provider 70. As shown in this example, the storage is a clinical trial data/research database 90 (on-going medical data, graphs, charts, etc, generated by the clinical trial), a clinical trial provider records database 100 (e.g., electronic health records (EHRs)/personal health records (PHRs) database), a clinical trial parameters database 110 (relevant data for analysis of clinical trial results based on trial parameters), and a clinical trial participant medical database 120 (personal health records, etc).

In operation, the clinical trial participant module 30 can allow for a clinical trial participant to upload health data by connecting health monitoring devices to internet enabled computers or cell phones, or by manually entering the data through a website. It can allow for a clinical trial participant to own his or her data and it can give the clinical trial participant access to a selected clinical trial provider to view or update the data, where and the clinical trial participant can revoke access anytime. The clinical trial participant module 30 can also suggest action to the clinical trial participants based on data. It further can allow for tracking of clinical trial compliance data for clinical trial participant reward programs, such as on successful completion of drug schedules. The clinical trial participant module 30 also can encourage and track social networking, such as through email, or text messaging from the system website between clinical trial participants and physicians, thereby helping in positive behavior modification, such as through lifestyle changes, diet, or exercise.

Each of the clinical trial provider module 40, in operation, allows for a clinical trial provider to have the EHR connected to the system to enable data exchange. A clinical trial provider can also use the clinical trial provider module store intelligent algorithms on the system for clinical trial participant treatment compliance. Further, a clinical trial provider can communicate with a clinical trial participant through the system, such as through a system website, by the use of a computer or cell phone. The clinical trial data module 50, in operation, can allow for clinical trial investigator to use a clinical trial data/research data repository to do statistical analysis and forecasts, or to recruit clinical trial participants for clinical trials.

### Example 2

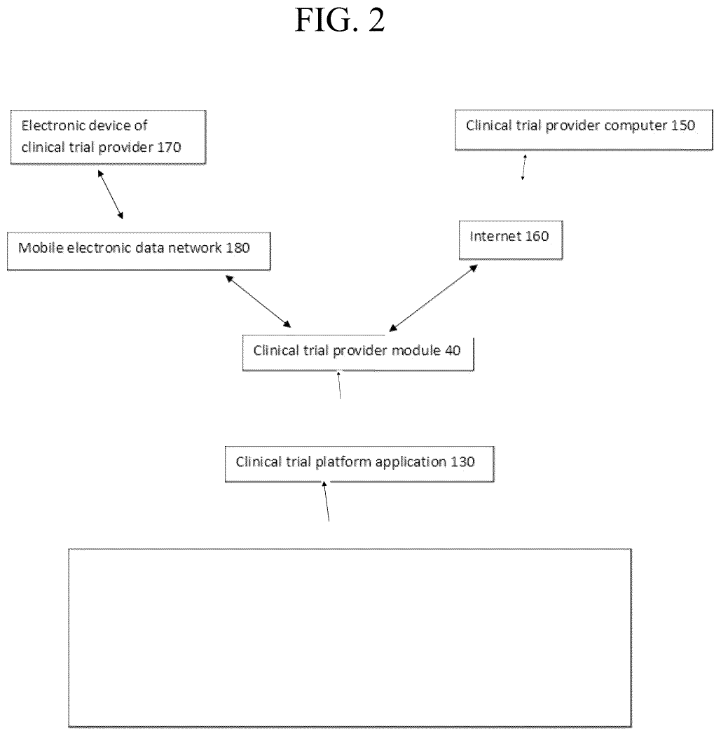

FIG. 2 shows schematically a further illustration of the clinical trial provider module 40 of the computer system 10 of FIG. 1, according to one embodiment of the present invention. As shown, the clinical trial provider module 40 is operatively associated with the clinical trial platform application 130, which receives and can also provide data to and from one or more clinical trial data monitoring devices or records 140 used by the clinical trial participant, and is connected in a data network to a clinical trial provider computer 150. The clinical trial platform application 130 can connect with the clinical trial provider module 40 either directly, such as at the point of care, or through a network connection such as the internet 160. Data can be received and provided by the clinical trial provider 70 in connection with the clinical trial provider module 40 either remotely, such as through a computer 150 connected to the internet 160, or a mobile electronic device 170 such as a cell phone or PDA that is connected through a mobile electronic device network 180, such as a wireless cell phone network. The clinical trial platform application 130 can also communicate with the clinical trial provider module 40 through the mobile electronic device network 180.

The clinical trial provider module provides analysis to support a decision that is ultimately made by the clinical trial provider in relation to the clinical trial participant, meaning that useful information is provided at the setting of the clinical encounter. Clinical trial data monitoring devices or records 140 can be provided in communication with the computer system 10, such that data to and from such devices can be transformed, such as into settings or functions implemented by particular devices that are directly in contact with the health condition of the clinical trial participant.

### Example 3

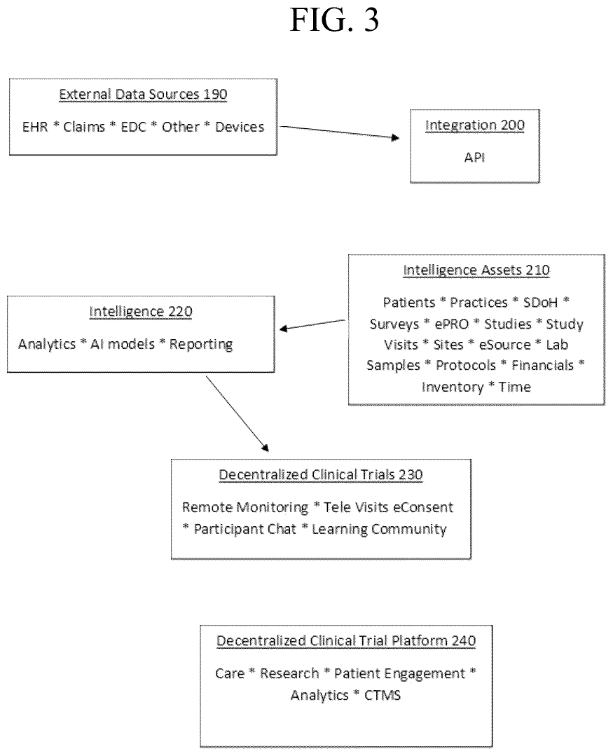

FIG. 3 shows schematically a further illustration of the clinical trial platform application 130 of FIG. 2, according to one embodiment of the present invention.

The table below shows clinical investigation platform features:

Clinical Investigation Platform Features

Recruit
EHR inclusion/exclusion
Contact Management
Activity Management
Email Campaigns
Call Center
CTMS
Study Management
Site Management
Financials Management
Subject Management
Visit Management
Protocols Management
Document Management
Intelligence
Interactive Reporting and Dashboard/Visuals
Power BI Reporting
Natural Language Query
Excel Import\Export
User Defined Views
Collaboration
Integrated Teams Chat
Integrated Teams Channels
Good Clinical Practice (GCP) Training
MS Office Productivity Suite
Outlook
Word
Excel
PowerPoint
Teams
Bookings
Planner
Resource Management
Lab Samples
Lab Inventory
Time Tracking
Service Requests
eSource
Protocol Defined Surveys
Protocol Defined Forms
Social determinants of health (SDoH)
User Defined Screens/dashboard
eConsent (electronic signature compliant with FDA 21 CFR Part 11)
Integrations
Platform Core
Health360x eSource
Document management and regulatory compliance
Electronic Trial Master File (eTMF)
Remote Access
Sponsors
Remote monitoring visits (CRO-Clinical Research Organization)
Study telehealth visits

External data sources 190 in the clinical setting may comprise at least electronic health records, medical claims (e.g., billing), clinical trial management system data (CTMS), data collected directly from the patient through monitoring devices, or other sources, such as clinical examinations, blood tests, etc. The data from these sources undergo integration 200 through an API and are turned into intelligence assets 210. Intelligence assets include, but are not limited to, individual patient records, records of practices in the clinical setting, records of social determinants of health (SDoH), survey records of clinical trial participants, data regarding electronic patient-reported outcomes (ePRO), studies of drug effects, results of study visits, site data, electronic source data, records of lab samples, protocols used for clinical testing or treatment, financial records, inventory records, and time records. The intelligence assets 210 undergo analysis by intelligence 220, which can include, but is not limited to, data analytics, artificial intelligence models, and data reporting. The product of the intelligence 220 is then fed into the decentralized clinical trials 230 which are being conducted by, for example, remote monitoring, tele visits, FDA 21 CFR Part 11 compliant electronic informed consent (eConsent), participant chat, or a learning community. The decentralized clinical trial platform 240 is wholly integrated with the decentralized clinical trials, so that patient care (e.g., clinical trial participant care), research, patient engagement (e.g., clinical trial participant engagement, including clinical trial eligibility decisions), and analytics, are managed and conducted through the clinical trial platform application 130.

### Example 4

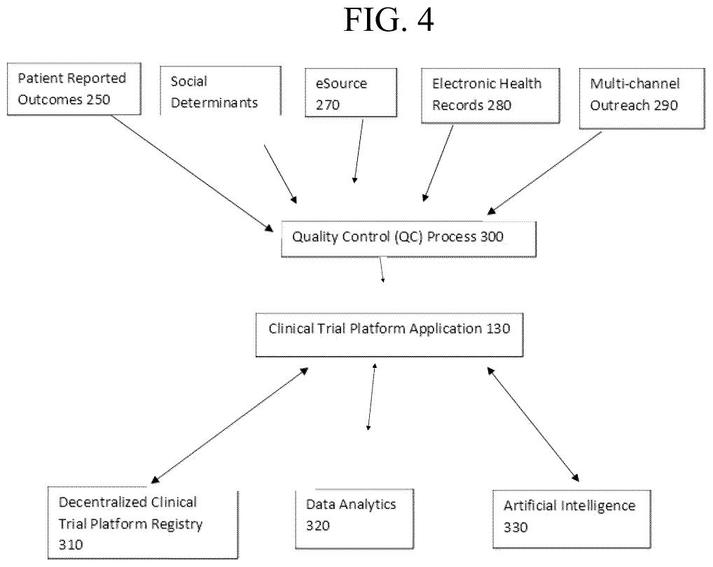

FIG. 4 shows schematically a further illustration of the clinical trial platform application 130 of FIG. 2, according to one embodiment of the present invention.

In certain embodiments, patient reported outcomes, 250, social determinants of health 260, electronic source data 270, electronic health records 280, and multi-channel outreach data 290, are subject to quality control process 300 before being entered into the clinical trial platform application 130.

The clinical trial platform application 130 also receives information from a decentralized clinical trial platform registry 310, data analytics 320, and artificial intelligence 330.

### Example 5

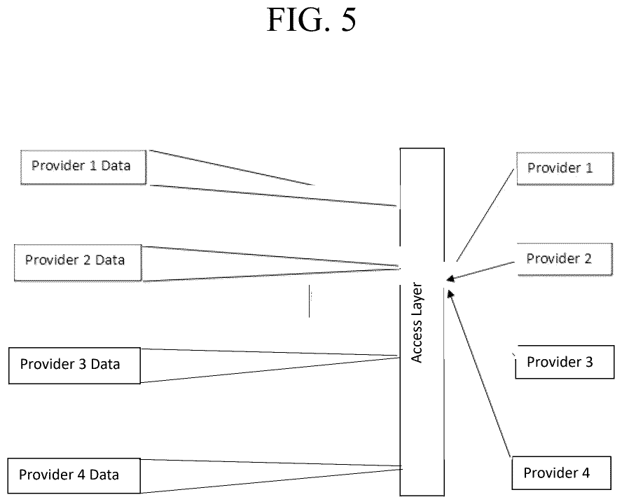

FIG. 5 shows schematically a further illustration of the clinical trial platform application 130 of FIG. 2, according to one embodiment of the present invention.

In certain embodiments, the clinical trial platform application provides each clinical trial provider with a view into their electronic database. Clinical trial provider electronic health record data is securely segregated to ensure only authorized and authenticated access to the decentralized clinical trial registry and the clinical trial provider database. All clinical trial platform data is persisted and transmitted in an encrypted format.

### Example 6

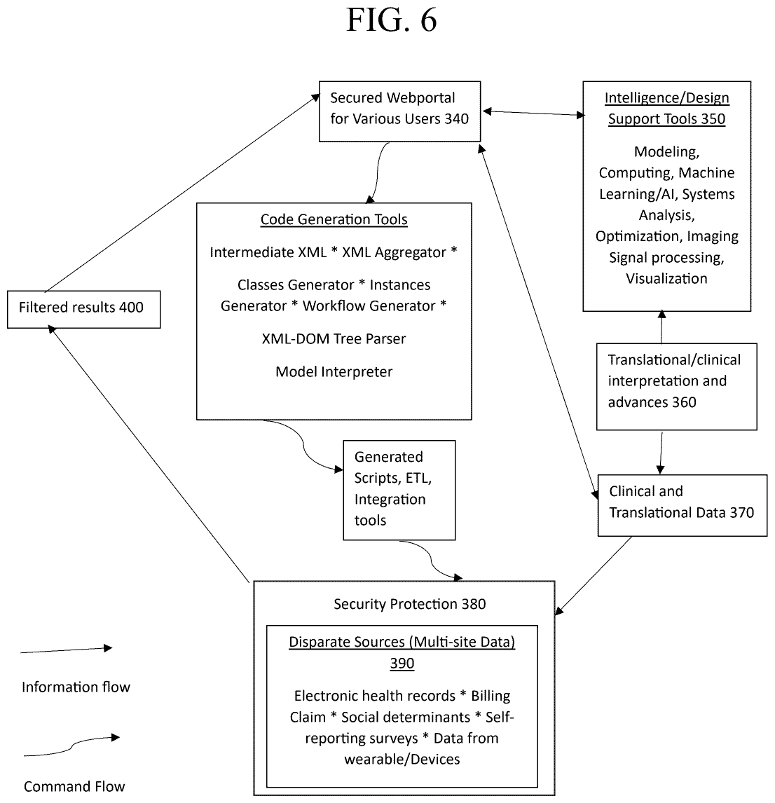

FIG. 6 shows schematically a further illustration of the clinical trial platform application 130 of FIG. 2, according to one embodiment of the present invention.

In certain embodiments, a secured webportal 340 is provided to users, which has information exchange with intelligence/design support tools 350. These intelligence/design support tools 350 exchange information with translational/clinical interpretations and advances 360 (e.g., inputs during clinical trial management); the product of the combined interchange of information yields clinical and translational data 370. The secured webportal 340 can also be used for direct entry of clinical and translational data 370. The clinical and translational data 370 is then subject to security protection 380, which encompasses disparate sources 390. After having been given security protection, filtered results 400 are provide to the secured webportal 340 where users can view them.

The present clinical trial platform application incorporates several important features: First, the clinical trial platform application is a purpose built, co-design HIPAA compliant software as a service (SaaS), that will incorporate a Role-based access control (RBAC) single sign-on, site facing platform designed to streamline workflow and reduce site burden. Second, AI/ML and natural language processing (NLP) of unstructured data will automate technology enabled EHR recruitment and other study processes such as inclusion/exclusion criteria, and matching cases to controls. Third, the clinical trial platform application Registry centers community partnered transparent data governance, with intelligent technologies for clinical research adaptive and “synthetic arms” study design. Fourth, Quality by design risk-based monitoring to improve trial efficiency, real time data quality control, and workflow integration, for seamless study management, and a transparent e-Consent process. Fifth, the clinical trial platform application protocol based training, and Virtual Collaboratory for continuous learning, will provide ongoing mentorship and peer to peer support to site clinical research coordinators. Sixth, the conceptual model integrates workflows for a care-to-research continuum, designed to improve clinical trial access for small to medium practices and the historically under-represented patient populations they serve.

The present clinical investigation/trial platform application has been implemented in a number of clinical studies in the African American, Hispanic and Rural White patient populations. Thousands of patients enrolled through over thirty community based practices, which would not be possible without the present clinical investigation/trial platform application.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives there intended, unless the context specifically indicates the contrary.

What is claimed is:

1. A method of using a computer system-programmed for providing access to at least one clinical trial participant participating in a clinical trial, comprising the steps of:
(a) accessing the computer system through a network, the computer system comprising a server comprising:
(i) a clinical trial provider module adapted for receiving, storing, and providing data in communication with at least one clinical trial provider at assessment of clinical trial eligibility, wherein the clinical trial provider module comprises an algorithm for generating at least one clinical trial eligibility decision for the at least one clinical trial participant at assessment of clinical trial eligibility, wherein the algorithm is based at least in part on inclusion criteria and exclusion criteria for the clinical trial;
(ii) a clinical trial participant module adapted for receiving, storing, and providing data in communication with the at least one clinical trial participant; and
(iii) a clinical trial data module adapted for receiving, storing, and providing data in communication with at least one clinical trial investigator, wherein the clinical trial data module is adapted for generating the inclusion criteria and exclusion criteria for the clinical trial,
wherein the server and each of the clinical trial provider module, clinical trial participant module, and clinical trial data module are operatively associated with a corresponding one of the at least one clinical trial provider, the at least one clinical trial participant, and the at least one clinical trial investigator through a network computing system to perform for interacting with at least one user of the system programmed for providing access to at least one clinical trial participant participating in at least one clinical trial;
(b) providing identification designating that the user is a clinical trial provider, clinical trial participant, or clinical trial investigator; and
(c) performing at least one of the steps of:
(i) providing data comprising at least one of information from electronic health records associated with the at least one clinical trial participant, information associated with at least one treatment compliance program for the at least one clinical trial, and information associated with the clinical target disease of the at least one clinical trial participant;
(ii) receiving an alert when a proposed medication or proposed medication dosage conflicts with clinical trial participant-specific treatment guidelines, clinical trial participant-specific medication plans, and clinical trial participant-specific care pathways associated with the at least one clinical trial participant;
(iii) receiving data for supporting clinical trial participant treatment compliance to clinical trial-specific treatment guidelines selected by at least one of the at least one clinical trial provider;
(iv) providing data through the network from at least one of a health monitoring device, mobile electronic device, and user computer;
(v) tracking use of clinical trial participant-specific forms by the at least one clinical trial participant;
(vi) tracking communication between each of the at least one clinical trial participant and at least one of another clinical trial participant, the clinical trial provider;
(vii) generating inclusion criteria and exclusion criteria associated with at least one clinical trial eligibility decision for a clinical trial selected by at least one of the at least one clinical trial provider;
(viii) analyzing clinical trial participant-specific data; and
(xi) receiving data associated with at least one clinical trial.

2. A method of using a computer system programmed for providing clinical to at least one clinical trial participant participating in a clinical trial, comprising the steps of:
(a) accessing the computer system through a network; the computer system comprising a server comprising:
(i) a clinical trial provider module adapted for receiving, storing, and providing data in communication with at least one clinical trial provider at assessment of clinical trial eligibility, wherein the clinical trial provider module comprises an algorithm for generating at least one clinical trial eligibility decision for the at least one clinical trial participant at assessment of clinical trial eligibility, wherein the algorithm is based at least in part on (1) inclusion criteria and exclusion criteria for the clinical trial;
(ii) a clinical trial participant module adapted for receiving, storing, and providing data in communication with the at least one clinical trial participant; and
(iii) a clinical trial data module adapted for receiving, storing, and providing data in communication with at least one clinical trial investigator, wherein the clinical trial data module is adapted for generating the inclusion criteria and exclusion criteria for the clinical trial,
wherein the server and each of the clinical trial provider module, clinical trial participant module, and clinical trial data module are operatively associated with a corresponding one of the at least one clinical trial provider, the at least one clinical trial participant, and the at least one clinical trial investigator through a network computing system to perform for interacting with at least one user of the system programmed for providing access to at least one clinical trial participant participating in at least one clinical trial;
(b) providing identification designating that the user is a clinical trial provider; and
(c) performing at least one of the steps of:
(i) providing data that comprises at least one of information from electronic health records associated with the at least one clinical trial participant, information associated with at least one treatment compliance program for the at least one clinical trial, and information associated with the clinical target disease of the at least one clinical trial participant; and
(ii) receiving an alert when a proposed medication or proposed medication dosage conflicts with clinical trial participant-specific treatment guidelines, clinical trial participant-specific medication plans, and clinical trial participant-specific care pathways associated with the at least one clinical trial participant.

3. The method of claim 2, further comprising the step of:
(d) generating at least one clinical trial eligibility decision for the at least one clinical trial participant with a clinical trial at assessment of clinical trial eligibility.

4. A computer system programmed for providing access to at least one clinical investigation participant participating in a clinical investigation, comprising a server comprising:
(a) a clinical investigation provider module adapted for receiving, storing, and providing data in communication with at least one clinical investigation provider at assessment of clinical investigation eligibility, wherein the clinical investigation provider module comprises an algorithm for generating at least one clinical investigation eligibility decision for the at least one clinical trial participant at assessment of clinical investigation eligibility, wherein the algorithm is based at least in part on inclusion criteria and exclusion criteria for the clinical investigation;
(b) a clinical investigation participant module adapted for receiving, storing, and providing data in communication with the at least one clinical investigation participant; and
(c) a clinical investigation data module adapted for receiving, storing, and providing data in communication with at least one clinical investigator, wherein the clinical investigation data module is adapted for generating the inclusion criteria and exclusion criteria for the clinical investigation,
wherein the server and each of the clinical investigation provider module, clinical investigation participant module, and clinical investigation data module are operatively associated with a corresponding one of the at least one clinical investigation provider, the at least one clinical investigation participant, and the at least one clinical investigator through a network computing system to perform for interacting with at least one user of the system programmed for providing access to at least one clinical investigation participant participating in the clinical investigation.

5. The system of claim 4, wherein the clinical investigation is a clinical trial and wherein the algorithm is further based on at least one of clinical trial participant-specific diagnostic guidelines, clinical trial participant-specific treatment guidelines, clinical trial participant-specific medication plans, and clinical trial participant-specific care pathways.

6. The system of claim 5, wherein the system is further programmed for performing at least one of the acts of:
(a) providing data comprising at least one of information from electronic health records associated with the at least one clinical trial participant, information associated with at least one treatment compliance program for the at least one clinical trial, and information associated with the clinical target disease of the at least one clinical trial participant;
(b) receiving an alert when a proposed medication or proposed medication dosage conflicts with clinical trial participant-specific treatment guidelines, clinical trial participant-specific medication plans, and clinical trial participant-specific care pathways associated with the at least one clinical trial participant;
(c) receiving data for supporting clinical trial participant treatment compliance to clinical trial-specific treatment guidelines selected by at least one of the at least one clinical trial provider;
(d) providing data through the network from at least one of a health monitoring device, mobile electronic device, and user computer;
(e) tracking use of clinical trial participant-specific forms by the at least one clinical trial participant;
(f) tracking communication between each of the at least one clinical trial participant and at least one of another clinical trial participant, the clinical trial provider;
(g) generating inclusion criteria and exclusion criteria associated with at least one clinical trial eligibility decision selected by at least one of the at least one clinical trial provider;
(h) analyzing clinical trial participant-specific data; and
(i) receiving data associated with at least one clinical health trial.

7. The system of claim 4, wherein each of the clinical investigation provider module, clinical investigation participant module, and clinical investigation data module is further operatively associated with at least one data storage means through the network, wherein the at least one data storage means is adapted for storing data saved by the at least one corresponding clinical investigation provider, at least one clinical investigation participant, and at least one clinical investigator.

8. The system of claim 4, wherein the at least one clinical investigation is concerning heart disease, diabetes, obesity, or asthma.

9. The system of claim 4, wherein the network is at least one of the internet and a mobile electronic device network.

10. The system of claim 4, wherein the at least one clinical investigation provider is a physician, physician assistant, nurse, or nurse practitioner.

11. The system of claim 4, wherein the data received by at least one of the clinical investigation provider module comprises at least one of information from electronic health records associated with the at least one clinical investigation participant, information associated with treatment compliance programs for the at least one clinical investigation, and information associated with the clinical target disease of the at least one clinical investigation participant.

12. The system of claim 5, wherein the clinical trial provider module is further adapted for providing an alert to the at least one clinical trial provider when a proposed medication or proposed medication dosage conflicts with clinical trial participant-specific treatment guidelines, clinical trial participant-specific medication plans, and clinical trial participant-specific care pathways associated with the at least one clinical trial participant.

13. The system of claim 4, wherein the data received by the clinical investigation participant module comprises information provided by the at least one clinical trial participant through the network from at least one of a health monitoring device, mobile electronic device, and user computer.

14. The system of claim 13, wherein the at least one health monitoring device is a heart rate monitor, blood pressure meter, peak flow meter, pulse oximeter, pedometer, weighing scale, or glucometer.

15. The system of claim 4, wherein the data received by the clinical investigation participant module comprises at least one social determinant of the clinical target disease of the at least one clinical trial participant.

16. The system of claim 15, wherein the at least one social determinant comprises at least one of racial, ethnic, religious, behavioral, linguistic, income and geography/location-related associated with the clinical target disease of the at least one clinical investigation participant.

17. The system of claim 4, wherein the clinical investigation participant module is further adapted for tracking use of clinical investigation participant-specific clinical endpoints by the at least one clinical investigation participant.

18. The system of claim 4, wherein the clinical investigation participant module is further adapted for tracking communication between the at least one clinical investigation participant and at least one of another clinical investigation participant, the at least one clinical investigation provider.

19. The system of claim 4, wherein each of the clinical investigation provider module, clinical investigation participant module, and clinical investigation data module is operatively associated with a graphical user interface comprising controls for selectively receiving, storing, and providing data in the system in response to an action by at least one of the at least one clinical investigation provider, the at least clinical investigation participant, and the at least one clinical investigator.

20. A software product comprising a non-transitory computer-readable medium in which computer program instructions are stored, said instructions, when executed by a computing system of claim 4, cause the computing system to perform the acts of:
(a) receiving, storing, and providing data in communication with at least one clinical trial provider;
(b) receiving, storing, and providing data in communication with at least one clinical trial participant participating in a clinical trial;
(c) receiving, storing, and providing data in communication with at least one clinical trial investigator;
(d) facilitating interactions between each of the at least one clinical trial provider, the at least one clinical trial participant, and the at least one clinical trial investigator; and
(e) providing access to the at least one clinical trial participant,
wherein the software comprises an algorithm for generating at least one clinical trial eligibility decision for the clinical trial for the at least one clinical trial participant at assessment of clinical trial eligibility and at least one social determinant of the clinical target disease of the at least one clinical trial participant, wherein the algorithm is based at least in part on (1) inclusion criteria and exclusion criteria for the clinical trial and (2) at least one of clinical trial participant-specific diagnostic guidelines, clinical trial participant-specific treatment guidelines, clinical trial participant-specific medication plans, and clinical trial participant-specific care pathways.
